(19) United States
(12) Patent Application Publication
(10) Pub. No.: US 2026/0026901 A1
(43) Pub. Date: Jan. 29, 2026

(54) PEG DRILL FOR ROBOTIC SURGICAL SHOULDER ARTHROPLASTY

(71) Applicant: Zimmer, Inc., Warsaw, IN (US)

(72) Inventor: Michael Mueller, Warsaw, IN (US)

(21) Appl. No.: 19/278,707

(22) Filed: Jul. 23, 2025

Related U.S. Application Data
(60) Provisional application No. 63/676,631, filed on Jul. 29, 2024.

(51) Int. Cl. A61B 34/30; A61B 17/16; A61B 34/10

(52) U.S. Cl. CPC A61B 34/30; A61B 17/1615; A61B 17/1626; A61B 34/10; A61B 2034/107; A61B 2034/305

(57) ABSTRACT

An end effector for a robotic surgical arm can include a housing, a drive shaft, a motor, and two or more drill heads. The housing can be securable to the robotic surgical arm, and the housing can define a cavity therein. The drive shaft can be located at least partially within the housing. The motor can be located at least partially within the housing or the robotic surgical arm, and the motor can be operable to rotate the drive shaft. The two or more drill heads can be supported by the housing and can be engaged with the drive shaft to be driven by the drive shaft to rotate together. The two or more drill heads can be configured to simultaneously form two or more bores in a bone.

### CLAIM OF PRIORITY

This application claims the benefit of U.S. Provisional Patent Application Ser. No. 63/676,631, filed on Jul. 29, 2024, the benefit of priority of which is claimed hereby, and which is incorporated by reference herein in its entirety.

### BACKGROUND

The present disclosure relates generally to apparatus and devices used during robotically-assisted surgical procedures, such as for robotic joint replacement procedures, or arthroplasty procedures. Often, during a robotically-assisted surgical procedure, one or more tools are used during the procedure. The tools can be connected to the robotic surgical arm, allowing the robotic surgical arm to use or manipulate the tools during the procedure. In some robotically-assisted procedures, cutting instruments can be guided or operated by a robotic arm, such as for creating or drilling one or more holes or bores in one or more bones of a human body.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 illustrates a perspective view of a robotic surgical system.

FIG. 2 illustrates a perspective view of a robotic surgical system.

FIG. 3 illustrates an isometric view of an end effector of a robotic surgical system.

FIG. 4 illustrates an isometric view of an end effector of a robotic surgical system.

FIG. 5 illustrates an isometric view of an end effector of a robotic surgical system.

FIG. 6 illustrates a cross-sectional view of an end effector of a robotic surgical system across indicators 6-6 of FIG. 4.

FIG. 7 illustrates a perspective view of an implant and bone.

FIG. 8 illustrates a schematic view of a method.

FIG. 9 is a block diagram illustrating an example of a machine upon which one or more examples may be implemented.

### DETAILED DESCRIPTION

Robotic assisted surgeries can be performed on various orthopedic components including hip, knee, and shoulder arthroplasties. Robotic assisted surgeries for full or partial shoulder arthroplasties are becoming more common. There are two main types of shoulder arthroplasties, reverse shoulder arthroplasties and anatomic shoulder arthroplasties. In a reverse shoulder arthroplasty, the humeral head is replaced with a cup or bearing surface and a head or projection is secured to the scapula, effectively reversing typical anatomic shoulder interactions. In an anatomic shoulder arthroplasty, a humeral head and scapular glenoid are replaced with components intended to imitate traditional anatomic interactions; however, robotic assisted surgeries for anatomic shoulder arthroplasties require more steps than a reverse shoulder arthroplasty and have therefore been more slowly adopted. For example, an anatomic shoulder arthroplasty will often require multiple steps to drill or ream the central peg and the peripheral pegs of an anatomic glenoid prosthesis.

The present disclosure helps to address these issues by reducing the steps required for the peripheral pegs into one single step. That is, three bores are often required to be drilled into the scapula around the glenoid to prepare to receive the glenoid implant. In the present disclosure, the robotic surgical arm can include an end effector that includes three drill heads configured to operate together to simultaneously drill the three holes or bores in the scapula around the glenoid, effectively reducing three or more procedures to one procedure.

The above discussion is intended to provide an overview of subject matter of the present patent application. It is not intended to provide an exclusive or exhaustive explanation of the invention. The description below is included to provide further information about the present patent application.

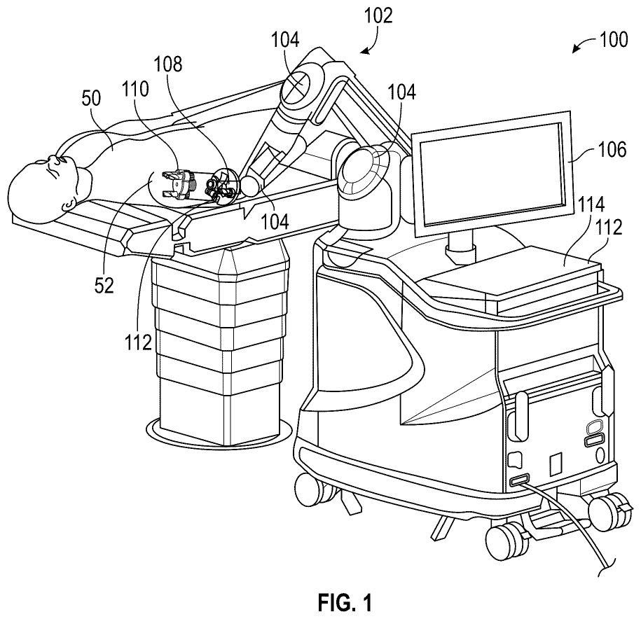

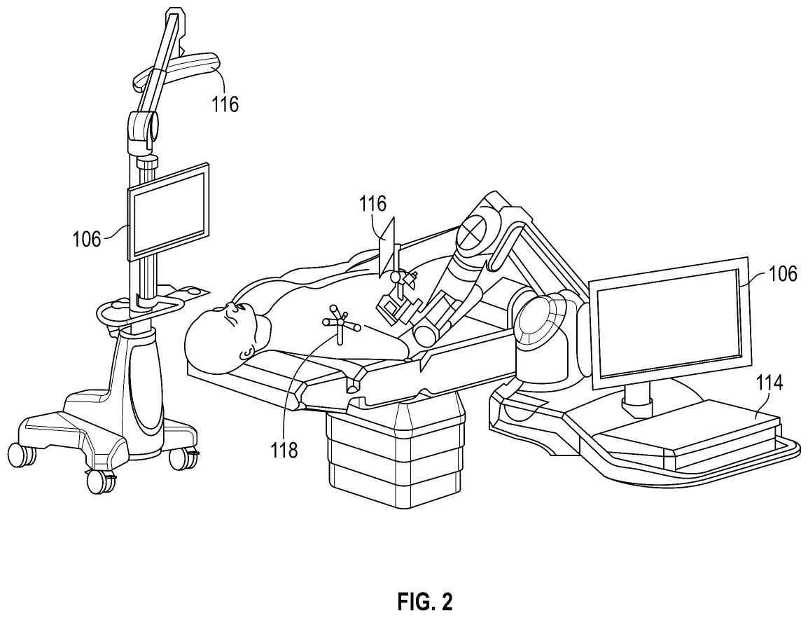

FIG. 1 illustrates a perspective view of a robotic surgical system 100 including a robotic surgical arm 102 and a patient 50. FIG. 2 illustrates a perspective view of the robotic surgical system 100 and the patient 50. FIG. 1 and FIG. 2 are discussed together below.

The robotic arm 102 can be a 6 degree-of-freedom (DOF) robot arm, such as the ROSA® robot from Medtech, a Zimmer Biomet Holdings, Inc. company. The robotic surgical arm 102 can be controlled by a surgeon with various control devices or systems. For example, a surgeon can use a control system (e.g., a controller that is processor-implemented based on machine-readable instructions, which when implemented cause the robotic arm to move automatically or to provide force assistance to surgeon-guided movement) to guide the robotic surgical arm 102. The robotic surgical arm 102 can include two or more articulating joints 104 capable of pivoting, rotating, or both, to provide a surgeon with wide range of adjustment options. A surgeon can also use anatomical imaging, such as displayed on a display screen 106, to guide and position the robotic surgical arm 102. Anatomical imaging can be provided to the display screen 106 with various imaging sources, or intraoperative fluoroscopy, such as a C-arm.

The anatomical imaging, for example, can be imaging of internal patient anatomy within an incision 52. The incision 52 can be made in a variety of positions on a patient. For example, in a shoulder arthroplasty procedure, the incision 52 can be made in a shoulder region of a patient. The incision 52 can be configured to allow one or more tools coupled to the robotic surgical arm 102 to access a bone surface, or other anatomy of the patient. The robotic surgical arm 102 can include a coupler 108 configured to connect an end effector 110 to the robotic surgical arm 102. The end effector 110 can include a base 112, which can be configured to connect the end effector 110 to the coupler 108 and therefore to the robotic surgical arm 102.

The robotic system 100 can include a computing system 114, which can also communicate with display screens 106 and a tracking system 116 (shown in FIG. 1B). The tracking system 116 can be operated by the computing system 114 as a stand-alone unit. The computing system 114 can optionally utilize the Polaris optical tracking system from Northern Digital, Inc. of Waterloo, Ontario, Canada. The tracking system 116 can monitor a plurality of tracking elements, such as tracking elements 118 (shown in FIG. 2). The tracking elements 118 can be affixed or connected to objects of interest (e.g., a bone or the end effector 110), to track locations of multiple objects within a surgical field.

The tracking system 116 can function to create a virtual three-dimensional coordinate system within the surgical field for tracking patient anatomy, surgical instruments, or portions of the end effector 110 or robotic surgical arm 102. The tracking element 118 can be tracking frames including multiple IR reflective tracking spheres, or similar optically tracked marker devices. In one example, the tracking element 118 can be placed on or adjacent one or more bones of patient. In other examples, the tracking element 118 can be placed on the end effector 110 and/or an implant to accurately track positions within the virtual coordinate system. In each instance the tracking element 118 can provide position data, such as a patient position, a bone position, a joint position, an implant position, a position of the robotic surgical arm 102, or the like.

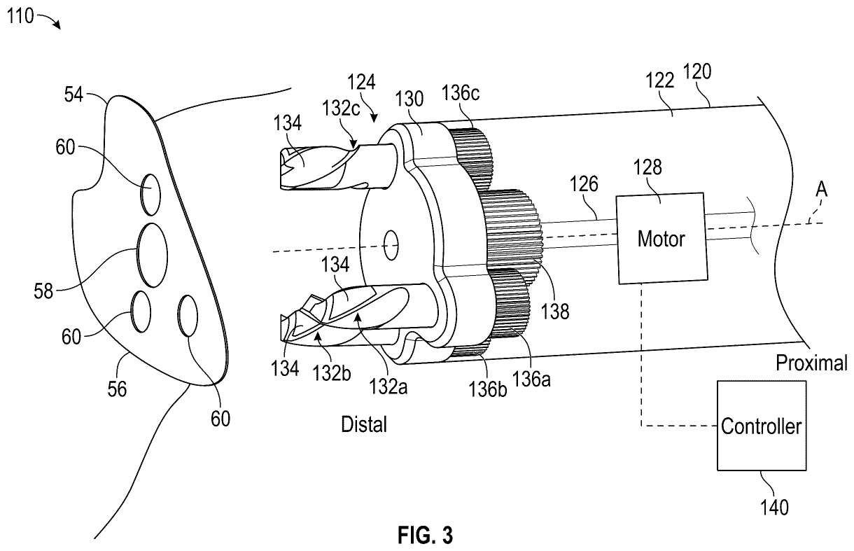

FIG. 3 illustrates an isometric view of the end effector 110 of the robotic surgical system 100 and a bone 54 or bones, such as a scapula, coracoid, glenoid 56, and acromion. The end effector 110 of FIG. 3 can be consistent with the end effector 110 discussed above with respect to FIG. 1 and FIG. 2; FIG. 3 shows additional details of the end effector 110.

The end effector 110 can include a housing 120 securable to the robotic surgical arm 102, such as via the base 112 that can be located at a proximal portion or proximal end of the housing 120, as shown in FIG. 1 above. The housing 120 can be a rigid or semi-rigid body made of materials such as one or more of metals, plastics, foams, elastomers, ceramics, composites, combinations thereof, or the like. The housing 120 can define a cavity 122 therein. The end effector 110 can also include a drill assembly 124 (which can be a peg drill assembly) that can be connected to a distal portion or distal end of the housing 120.

The drill assembly 124 can be connected to a drive shaft 126 located at least partially within the housing 120. The drive shaft 126 can be one or more shafts, rods, or the like configured to rotate within the housing 120. The drive shaft 126 can be connected to a motor 128 that can be located at least partially within the housing 120 or within the robotic surgical arm 102. The motor 128 can be connected (e.g., electrically) to the computing system 114, such that the computing system 114 can control the motor 128. The motor 128 can be operable to rotate the drive shaft 126, as discussed in further detail below.

The drill assembly 124 can include an end plate 130 that can be connected to the distal portion of the housing 120. Optionally, the end plate 130 can be integrally formed with the housing 120. The drill assembly 124 can also include drill heads 132 including a drill head 132a, a drill head 132b, and a drill head 132c. Though the drill assembly 124 shows three drill heads, the drill assembly 124 can include 1, 2, 4, 5, 6, 7, 8, or the like drill heads. Each of the drill heads 132 can include a drill bit 134 and a driven gear 136a. Each of the drill heads 132 can be supported by the housing 120 and can be configured to extend at least partially through the housing 120, such as through bores of the end plate 130. Each of the drill bits 134 can extend at least partially through the end plate 130 and can be configured to create, e.g., independently, a bore or hole in a bone. The drill bits 134 can each be made of one or more metals capable of forming bone bores, such as steel alloys, titanium, or the like. Optionally, the end plate 130 can be releasably coupled to the housing 120, such that the end plate 130 is removable from the housing 120, which can allow the drill heads 132 to be removed from the housing 120, such as for cleaning following a procedure.

The drill assembly 124 can also include a drive gear 138 connected to a distal portion or distal end of the drive shaft 126 such that the drive gear 138 is rotatable together with the drive shaft 126, e.g., as driven by the motor 128. The drive gear 138 can be engaged or interfaced with each of the driven gears 136a such that rotation of the drive gear 138 results in simultaneous rotation of the driven gears 136a, such as to form three holes in a bone for implantation of a shoulder implant.

The driven gears 136a and the drive gear 138 can be spur gears as shown or can be other types of gears such as bevel gears or helical gears. In some examples, the driven gears 136a can be a first type of gear and the drive gear 138 can be a different type of gear. For example, the driven gears 136a can be spur gears and the drive gear 138 can be a worm gear. Though the driven gears 136a are connected to the drill bits 134, the drill bits 134 and the driven gears 136a can be made of different metals. For example, the drill bits 134 can be made of titanium, and the driven gears 136a can be made of a steel alloy. In such an example, the drill bits 134 can be respectively connected (e.g., welded, fastened, or adhered) to the driven gears 136a. Optionally, the drill bits 134 and the driven gears 136a can be made of the same material or can be formed of a single piece.

FIG. 3 also shows that the end effector 110 can include a controller 140, which can be connected to or in communication with the motor 128. The controller 140 can be or can be part of the computing system 114 and can be in communication with various components of the robotic surgical arm 102. The controller 140 can be a programable controller, such as a single or multi-board computer, a direct digital controller (DDC), a programable logic controller (PLC), printed circuit board (PCB), or the like. In other examples the controller 140 can be any computing device, such as a handheld computer, for example, a smart phone, a tablet, a laptop, a desktop computer, or any other computing device including a processor, memory, and communication capabilities.

In operation of some examples, the controller 140 can be configured to align the drill assembly 124 with a bone based on a pre-operative plan and based on alignment between a central axis A and a central bore 58 formed in the bone 54, such as the glenoid 56. When the end effector 110 is properly aligned, as determined by the controller 140 (or the computing system 114), the controller 140 can control the robotic surgical arm 102 to advance the end effector 110 such that the drill bits 134 engage the bone 54. The controller 140 can then operate the motor 128 to drive the 126 to rotate (e.g., about the central axis A), which can cause the drive gear 138 to rotate with the drive shaft 126. Rotation of the drive gear 138 can drive the driven gears 136a to rotate simultaneously, such that the drill bits 134 rotate together to form, simultaneously or near simultaneously (e.g., during a single operation, maneuver, or step), the peg bores 60 in the bone 54. The peg bores 60 can be located in or around the glenoid 56 and around the central axis A, and the peg bores 60 can be configured to receive three pegs, respectively, of a glenoid component of an anatomical shoulder implant assembly. In this way the robotic surgical arm 102 can be used to simultaneously form three bores in the bone 54, which can save a significant number of steps in an overall procedure of preparing the bone 54 to receive a glenoid implant of an anatomical shoulder implant assembly.

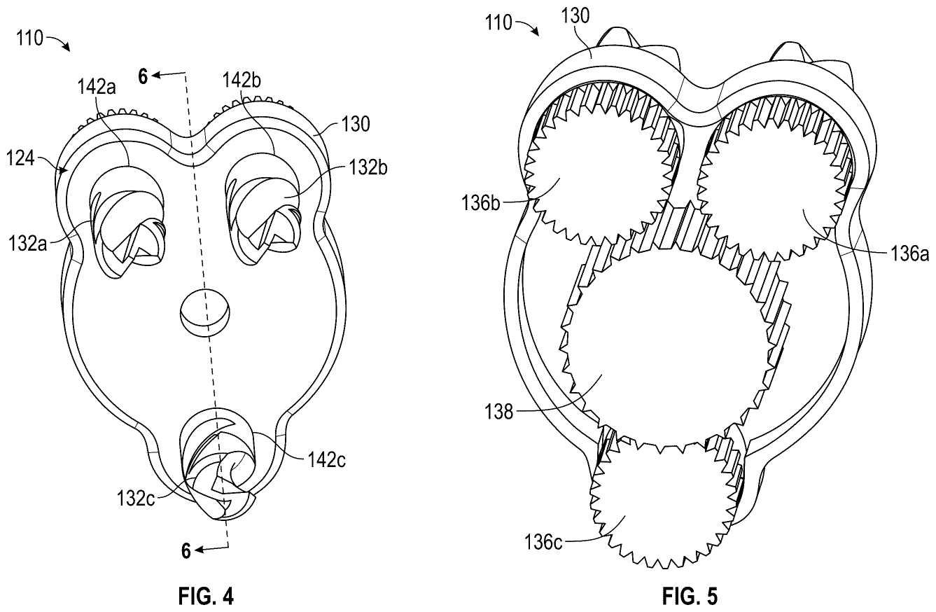

FIG. 4 illustrates an isometric view of the end effector 110 of the robotic surgical arm 102. The robotic surgical arm 102 of FIG. 4 can be consistent with the end effector 110 of FIG. 1-FIG. 3. FIG. 4 shows additional details of the end effector 110. For example, FIG. 4 shows that the end plate 130 can include or can define plate bores 142, which can extend through a distal face or portion of the end plate 130. The drill head 132a, drill head 132b, and drill head 132c can be configured to extend through plate bore 142a, plate bore 142b, and plate bore 142c, respectively, when the end effector 110 is assembled.

FIG. 5 illustrates an isometric view of the end effector 110 of the robotic surgical arm 102. The robotic surgical arm 102 of FIG. 5 can be consistent with the end effector 110 of FIG. 1-FIG. 4. FIG. 5 shows additional details of the end effector 110. For example, FIG. 5 shows how the driven gear 136a, the driven gear 136b, and the driven gear 136c, can each mesh or engage the drive gear 138, which can allow for the drive gear 138 to simultaneously drive or rotate the driven gears 136 to drive the drill bits 134 together. FIG. 5 also show that the driven gears 136 and the drive gear 138 can be different sizes, creating a gear ratio between the driven gears 136 and the 138. The gear ratio between the driven gears 136 and the drive gear 138 can be between XYZ and XYZ or can be between XYZ and XYZ. This can produce a cutting speed of the drill bits 134 of between XYZ and XYZ.

FIG. 5 also shows how the end plate 130 (and the housing 120) can be sized or shaped to minimize an outer dimension of the end effector 110. For example, the end plate 130 (and the housing 120) can conform to the driven gears 136 such that the size of the housing 120 and end plate 130 is minimized, which can help reduce interference of the end effector 110 with other instruments during a procedure.

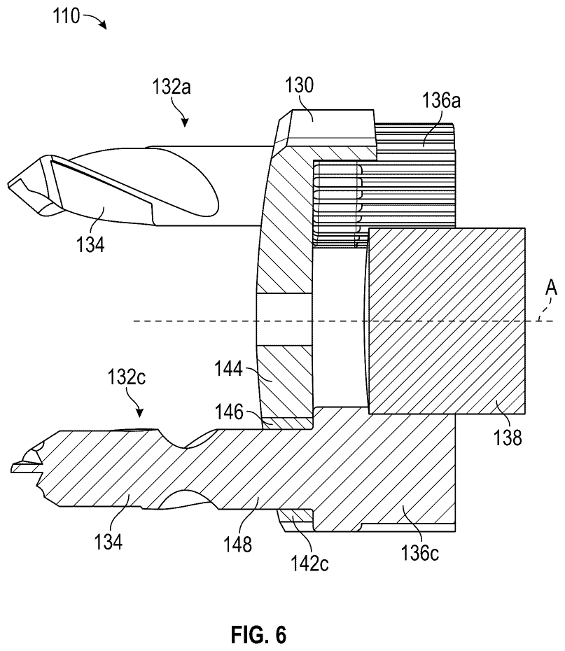

FIG. 6 illustrates a cross-sectional view of an end effector of a robotic surgical system across indicators 6-6 of FIG. 4. The robotic surgical arm 102 of FIG. 6 can be consistent with the end effector 110 of FIG. 1-FIG. 5. FIG. 6 shows additional details of the end effector 110. For example, FIG. 6 more clearly shows that the drill heads 132 can define the central axis A therebetween.

FIG. 6 also shows how the plate bores 142 (e.g., the plate bore 142c) can extend through a distal portion 144 of the end plate 130. FIG. 6 also shows that the end effector 110 can include a bushing 146 connected to the end plate 130 and located at least partially in each of the plate bores 142. The bushings 146 can be configured to interface with a shank 148 (or shaft) of each of the drill bit 134 and can help to reduce friction between the drill bit 134 and the end plate 130. The bushings 146 can be affixed to the end plate 130, such as pressed, welded, or adhered to the end plate 130. Optionally, the bushings 146 can be a bearing, such as a journal bearing or ball bearing. The bushings 146 can be lubricated or can be comprised of self-lubricating materials such as lubricant-impregnated phosphor bronze. Optionally, the bushings 146 can be removable and replaceable.

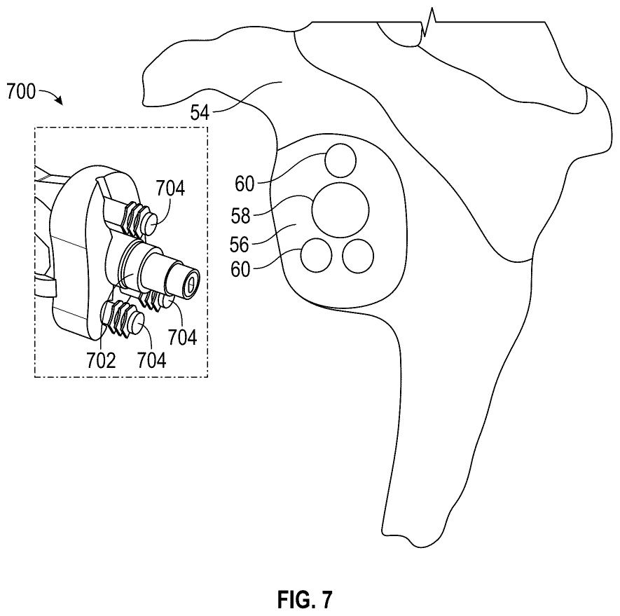

FIG. 7 illustrates a perspective view of an implant 700 and the bone 54, which can include the glenoid 56. FIG. 7 shows that the implant 700 can include a stem 702, which can be a central stem of the implant 700, and can include pegs 704, which can can be peripheral pegs, arms, or projections of the implant 700. The stem 702 can be insertable into the central bore 58 and the pegs 704 can be insertable into the peg bores 60, such as to secure the implant 700 of the glenoid 56.

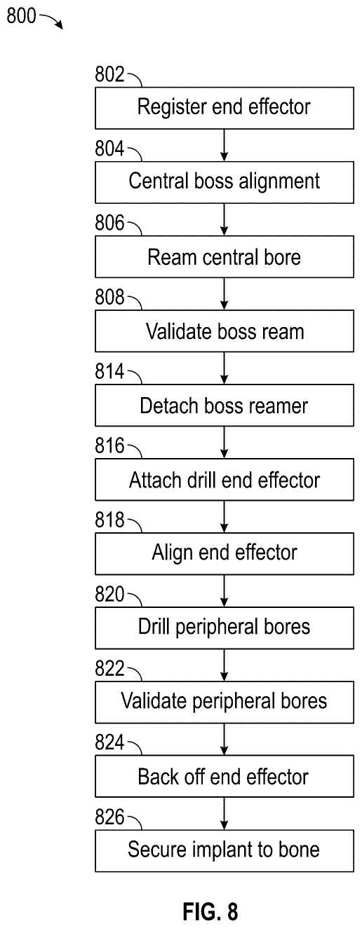

FIG. 8 illustrates a schematic view of a method 800. The method 800 can be a method of installing one or more portions of an implant using a robotic surgical arm. The steps or operations of the method 800 are illustrated in a particular order for convenience and clarity; many of the discussed operations can be performed in a different sequence or in parallel without materially impacting other operations. The steps or operations of the method 800 can be omitted or can be performed multiple times. The method 800 as discussed includes operations that can be performed by multiple different actors, devices, and/or systems. It should be understood that subsets of the operations discussed in the method 800 can be attributable to a single actor, device, or system could be considered a separate standalone process or method. Prior to or during the method 800, a preoperative plan can be uploaded (or developed on) the computing system 114 or another computing system and the computing system 114 can use the preoperative plan or can receive portions of the preoperative plan from a remote computing system.

The method 800 can begin at step 802, where the end effector can be registered. For example, the end effector 110 can be registered using one or more tracking element 118 by the tracking system 116 and the computing system 114. Optionally, after the step 802, a glenoid surface can be reamed, such as using a bow-tie ream. At step 804, a boss reamer can be aligned with a central point of the glenoid 56, such as based on the preoperative (or perioperative plan). At step 806, a central bore can be reamed using the bore reamer and the surgical arm and the reamer can be disengaged from the glenoid. For example, the robotic surgical arm 102 can operate the boss reamer or bore reamer to form the central bore 58 in the glenoid 56. At step 808, the central bore can be validated. For example, the tracking system 116 and the computing system 114 can be used to validate a location and size of the central bore 58, such as based on the central bore 58 and the preoperative surgical plan. At step 814, the reamer can be detached from the robotic surgical arm. For example, the reamer or end effector can be detached from the 102.

At step 816, a drill can be connected to the robotic surgical arm. For example, the end effector 110 can be connected to the 102. At step 818, the end effector or drill can be aligned with the glenoid. For example, the end effector 110 (e.g., including the drill heads 132) can be aligned with the glenoid 56 based on the preoperative surgical plan and based on alignment between the end effector 110, the central axis A, and the central bore 58 formed in the glenoid 56. At step 820, the peripheral bores can be formed or drilled. For example, the end effector 110 (e.g., including the drill heads 132) can be used to, simultaneously (or nearly simultaneously) form the peg bores 60 in the glenoid 56. At step 822, the peripheral bores can be validated. For example, the tracking system 116 and the computing system 114 can be used to validate a location and size of each of the peg bores 60, such as based on the peg bores 60 and the preoperative surgical plan. For example, the peg bores 60 and the central bore 58 can be compared with virtual peg drill holes and a virtual central bore of the preoperative surgical plan. At step 824, the end effector can be retracted or backed off the bone. For example, the robotic surgical arm 102 can be operated to retract or move the end effector 110 away from the glenoid 56 such that the end effector 110 disengages the glenoid 56. At step 826, the implant can be secured to the bone. For example, the implant 700 can be secured to the glenoid 56, such as by inserting the stem 702 into the central bore 58 and by inserting the pegs 704 into the peg bores 60.

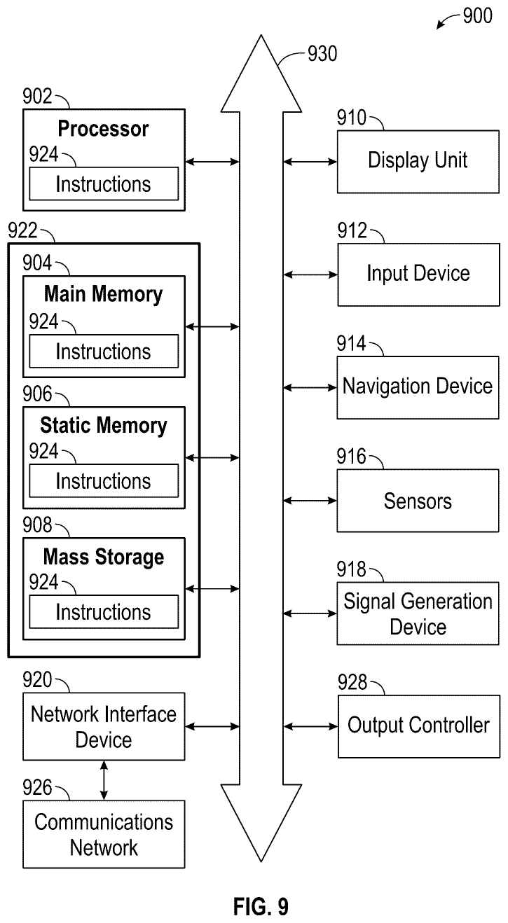

FIG. 9 illustrates a block diagram of an example machine 900 upon which any one or more of the techniques (e.g., methodologies) discussed herein may perform. Examples, as described herein, may include, or may operate by, logic or a number of components, or mechanisms in the machine 900. Circuitry (e.g., processing circuitry) is a collection of circuits implemented in tangible entities of the machine 900 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.), including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 900 follow.

In alternative examples, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 900 may include a hardware processor 902 (e.g., a central processing unit (CPU), a graphics processing unit (GPU), a hardware processor core, or any combination thereof), a main memory 904, a static memory (e.g., memory or storage for firmware, microcode, a basic-input-output (BIOS), and mass storage 908 (e.g., hard drives, tape drives, flash storage, or other block devices) some or all of which may communicate with each other via an interlink 930 (e.g., bus). The machine 900 may further include a display unit 910, an alphanumeric input device 912 (e.g., a keyboard), and a user interface (UI) navigation device 914 (e.g., a mouse). In examples, the display unit 910, input device 912 and UI navigation device 914 may be a touch screen display. The machine 900 may additionally include a signal generation device 918 (e.g., a speaker), a network interface device 920, and one or more sensors 916, such as a global positioning system (GPS) sensor, compass, accelerometer, or other sensor. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 902, the main memory 904, the static memory 906, or the mass storage 908 may be, or include, a machine-readable medium 922 on which is stored one or more sets of data structures or instructions 924 (e.g., software) embodying or utilized by any one or more of the techniques or functions described herein. The instructions 924 may also reside, completely or at least partially, within any of registers of the processor 902, the main memory 904, the static memory 906, or the mass storage 908 during execution thereof by the machine 900. In an example, one or any combination of the hardware processor 902, the main memory 904, the static memory 906, or the mass storage 908 may constitute the machine-readable media 922. While the machine-readable medium 922 is illustrated as a single medium, the term “machine-readable medium” may include a single medium or multiple media (e.g., a centralized or distributed database, and/or associated caches and servers) configured to store the one or more instructions 924.

The term “machine-readable medium” may include any medium that is capable of storing, encoding, or carrying instructions for execution by the machine 900 and that cause the machine 900 to perform any one or more of the techniques of the present disclosure, or that is capable of storing, encoding or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine-readable medium 922 may be representative of the instructions 924, such as instructions 924 themselves or a format from which the instructions 924 may be derived. This format from which the instructions 924 may be derived may include source code, encoded instructions (e.g., in compressed or encrypted form), packaged instructions (e.g., split into multiple packages), or the like. The information representative of the instructions 924 in the machine-readable medium 922 may be processed by processing circuitry into the instructions to implement any of the operations discussed herein. For example, deriving the instructions 924 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 924.

In an example, the derivation of the instructions 924 may include assembly, compilation, or interpretation of the information (e.g., by the processing circuitry) to create the instructions 924 from some intermediate or preprocessed format provided by the machine-readable medium 922. The information, when provided in multiple parts, may be combined, unpacked, and modified to create the instructions 924. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 924 may be further transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 802.11 family of standards known as Wi-Fi®, IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 900, and includes digital or analog communications signals or other intangible medium to facilitate communication of such software. A transmission medium is a machine-readable medium.

### NOTES AND EXAMPLES

The following, non-limiting examples, detail certain aspects of the present subject matter to solve the challenges and provide the benefits discussed herein, among others.

Example 1 is an end effector for a robotic surgical arm, the end effector comprising: a housing securable to the robotic surgical arm, the housing defining a cavity therein; a drive shaft located at least partially within the housing; a motor located at least partially within the housing or the robotic surgical arm, the motor operable to rotate the drive shaft; two or more drill heads supported by the housing and engaged with the drive shaft to be driven by the drive shaft to rotate together, the two or more drill heads configured to simultaneously form two or more bores in a bone.

In Example 2, the subject matter of Example 1 optionally includes a drive gear connected to a distal end portion of the drive shaft, the drive gear configured to simultaneously drive the two or more drill heads to rotate.

In Example 3, the subject matter of Example 2 optionally includes wherein each of the two or more drill heads include: a drill bit extending at least partially from the housing; and a driven gear connected to the drill bit, each driven gear located at least partially within the housing, each driven gear interfaced with the drive gear, and each driven gear drivable to rotate by the drive gear to rotate the drill bit to form a bore in the bone.

In Example 4, the subject matter of any one or more of Examples 1-3 optionally include wherein the two or more drill heads includes three drill heads, the three drill heads defining a central axis therebetween.

In Example 5, the subject matter of Example 4 optionally includes a controller connected to the motor and the robotic surgical arm, the controller configured to: align the two or more drill heads with the bone based on a pre-operative plan and based on alignment between the central axis and a central bore formed in the bone.

In Example 6, the subject matter of Example 5 optionally includes wherein the three drill heads are configured to, together, form three peripheral peg drill holes in or around a glenoid and around the central axis, the three peripheral peg drill holes configured to receive three pegs, respectively, of a glenoid component of an anatomical shoulder implant assembly.

In Example 7, the subject matter of any one or more of Examples 1-6 optionally include wherein the housing includes a first portion and a second portion, the second portion releasable from the first portion to allow each of the two or more drill heads to be removed from the housing.

Example 8 is an end effector for a robotic surgical arm, the end effector comprising: a housing securable to the robotic surgical arm, the housing defining a cavity therein; a drive shaft located at least partially within the housing; a motor located at least partially within the housing or the robotic surgical arm, the motor operable to rotate the drive shaft; three drill heads defining a central axis therebetween, the three drill heads supported by the housing and engaged with the drive shaft to be together driven by the drive shaft to rotate together, the three drill heads configured to simultaneously form three or more bores in a bone; a controller connected to the motor and the robotic surgical arm, the controller configured to: operate the robotic surgical arm and the end effector to align the end effector and the three drill heads with the bone based on a pre-operative plan and based on alignment between the central axis and a central bore formed in the bone.

In Example 9, the subject matter of Example 8 optionally includes a drive gear connected to a distal end portion of the drive shaft, the drive gear configured to simultaneously drive the three drill heads to rotate.

In Example 10, the subject matter of Example 9 optionally includes wherein each of the three drill heads include: a drill bit extending at least partially from the housing; and a driven gear connected to the drill bit, each driven gear located at least partially within the housing, each driven gear interfaced with the drive gear, and each driven gear drivable to rotate by the drive gear to form a bore in the bone.

In Example 11, the subject matter of Example 10 optionally includes wherein the controller is configured to: operate the robotic surgical arm and the end effector to rotate the three drill heads together to, simultaneously, rotate the drill bit of each of the three drill heads to form three holes in the bone.

In Example 12, the subject matter of Example 11 optionally includes wherein the three drill heads are configured to, together, form three peripheral peg drill holes in or around a glenoid and around the central axis, the three peripheral peg drill holes configured to receive three pegs, respectively, of a glenoid component of an anatomical shoulder implant assembly.

Example 13 is a method of operating a robotic surgical arm, the method comprising: aligning a reamer connected to an end effector the robotic surgical arm with a glenoid of a bone according to a preoperative surgical plan; operating the robotic surgical arm and the reamer to form a central bore along a central axis in the glenoid; disengaging the reamer from the glenoid; detaching the reamer from the robotic surgical arm; securing a peg drill assembly to the robotic surgical arm, the peg drill assembly including three drill heads; aligning the end effector and the three drill heads with the glenoid based on the preoperative surgical plan and based on alignment between the peg drill assembly, the central axis, and the central bore formed in the glenoid; and drilling three peripheral peg drill holes, simultaneously, using the peg drill assembly.

In Example 14, the subject matter of Example 13 optionally includes moving the robotic surgical arm to disengage the three drill heads from the glenoid.

In Example 15, the subject matter of any one or more of Examples 13-14 optionally include validating the three peripheral peg drill holes by comparing a location of each of the three peripheral peg drill holes to the preoperative surgical plan.

In Example 16, the subject matter of Example 15 optionally includes inserting a glenoid trial component of an anatomical shoulder implant assembly into the glenoid of the bone.

In Example 17, the subject matter of any one or more of Examples 13-16 optionally include validating the three peripheral peg drill holes and the central bore by comparing a location of each of the three peripheral peg drill holes and the central bore to locations of virtual peg drill holes and a virtual central bore of the preoperative surgical plan.

In Example 18, the subject matter of any one or more of Examples 13-17 optionally include wherein operating the reamer includes operating a motor within one or more of the end effector and the robotic surgical arm.

In Example 19, the subject matter of Example 18 optionally includes wherein the three drill heads are connected to the motor via a drive shaft located at least partially within the housing.

In Example 20, the subject matter of Example 19 optionally includes wherein each of the three drill heads is connected to a driven gear that are each interfaced with a drive gear, the drive gear connected to the drive shaft.

In Example 21, the apparatuses or method of any one or any combination of Examples 1-20 can optionally be configured such that all elements or options recited are available to use or select from.

The above detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. An end effector for a robotic surgical arm, the end effector comprising:
a housing securable to the robotic surgical arm, the housing defining a cavity therein;
a drive shaft located at least partially within the housing;
a motor located at least partially within the housing or the robotic surgical arm, the motor operable to rotate the drive shaft;
two or more drill heads supported by the housing and engaged with the drive shaft to be driven by the drive shaft to rotate together, the two or more drill heads configured to simultaneously form two or more bores in a bone.

2. The end effector of claim 1, comprising:
a drive gear connected to a distal end portion of the drive shaft, the drive gear configured to simultaneously drive the two or more drill heads to rotate.

3. The end effector of claim 2, wherein each of the two or more drill heads include:
a drill bit extending at least partially from the housing; and
a driven gear connected to the drill bit, each driven gear located at least partially within the housing, each driven gear interfaced with the drive gear, and each driven gear drivable to rotate by the drive gear to rotate the drill bit to form a bore in the bone.

4. The end effector of claim 1, wherein the two or more drill heads includes three drill heads, the three drill heads defining a central axis therebetween.

5. The end effector of claim 4, comprising:
a controller connected to the motor and the robotic surgical arm, the controller configured to:
align the two or more drill heads with the bone based on a pre-operative plan and based on alignment between the central axis and a central bore formed in the bone.

6. The end effector of claim 5, wherein the three drill heads are configured to, together, form three peripheral peg drill holes in or around a glenoid and around the central axis, the three peripheral peg drill holes configured to receive three pegs, respectively, of a glenoid component of an anatomical shoulder implant assembly.

7. The end effector of claim 1, wherein the housing includes a first portion and a second portion, the second portion releasable from the first portion to allow each of the two or more drill heads to be removed from the housing.

8. An end effector for a robotic surgical arm, the end effector comprising:
a housing securable to the robotic surgical arm, the housing defining a cavity therein;
a drive shaft located at least partially within the housing;
a motor located at least partially within the housing or the robotic surgical arm, the motor operable to rotate the drive shaft;
three drill heads defining a central axis therebetween, the three drill heads supported by the housing and engaged with the drive shaft to be together driven by the drive shaft to rotate together, the three drill heads configured to simultaneously form three or more bores in a bone;
a controller connected to the motor and the robotic surgical arm, the controller configured to:
operate the robotic surgical arm and the end effector to align the end effector and the three drill heads with the bone based on a pre-operative plan and based on alignment between the central axis and a central bore formed in the bone.

9. The end effector of claim 8, comprising:
a drive gear connected to a distal end portion of the drive shaft, the drive gear configured to simultaneously drive the three drill heads to rotate.

10. The end effector of claim 9, wherein each of the three drill heads include:
a drill bit extending at least partially from the housing; and
a driven gear connected to the drill bit, each driven gear located at least partially within the housing, each driven gear interfaced with the drive gear, and each driven gear drivable to rotate by the drive gear to form a bore in the bone.

11. The end effector of claim 10, wherein the controller is configured to:
operate the robotic surgical arm and the end effector to rotate the three drill heads together to, simultaneously, rotate the drill bit of each of the three drill heads to form three holes in the bone.

12. The end effector of claim 11, wherein the three drill heads are configured to, together, form three peripheral peg drill holes in or around a glenoid and around the central axis, the three peripheral peg drill holes configured to receive three pegs, respectively, of a glenoid component of an anatomical shoulder implant assembly.

13. A method of operating a robotic surgical arm, the method comprising:
aligning a reamer connected to an end effector the robotic surgical arm with a glenoid of a bone according to a preoperative surgical plan;
operating the robotic surgical arm and the reamer to form a central bore along a central axis in the glenoid;
disengaging the reamer from the glenoid;
detaching the reamer from the robotic surgical arm;
securing a peg drill assembly to the robotic surgical arm, the peg drill assembly including three drill heads;
aligning the end effector and the three drill heads with the glenoid based on the preoperative surgical plan and based on alignment between the peg drill assembly, the central axis, and the central bore formed in the glenoid; and
drilling three peripheral peg drill holes, simultaneously, using the peg drill assembly.

14. The method of claim 13, comprising:
moving the robotic surgical arm to disengage the three drill heads from the glenoid.

15. The method of claim 13, comprising:
validating the three peripheral peg drill holes by comparing a location of each of the three peripheral peg drill holes to the preoperative surgical plan.

16. The method of claim 15, comprising:
inserting a glenoid trial component of an anatomical shoulder implant assembly into the glenoid of the bone.

17. The method of claim 13, comprising:
validating the three peripheral peg drill holes and the central bore by comparing a location of each of the three peripheral peg drill holes and the central bore to locations of virtual peg drill holes and a virtual central bore of the preoperative surgical plan.

18. The method of claim 13, wherein operating the reamer includes operating a motor within one or more of the end effector and the robotic surgical arm.

19. The method of claim 18, wherein the three drill heads are connected to the motor via a drive shaft located at least partially within the housing.

20. The method of claim 19, wherein each of the three drill heads is connected to a driven gear that are each interfaced with a drive gear, the drive gear connected to the drive shaft.
